(19) United States
(12) Patent Application Publication
(10) Pub. No.: US 2023/0302226 A1
(43) Pub. Date: Sep. 28, 2023

(54) PREFILLED SYRINGES WITH DIFFERENT INJECTION FORCES

(71) Applicant: Hongdan Chen, BELMONT, MA (US)

(72) Inventor: Hongdan Chen, BELMONT, MA (US)

(21) Appl. No.: 17/656,649

(22) Filed: Mar. 27, 2022

(51) Int. Cl. A61M 5/315

(52) U.S. Cl. CPC A61M 5/315

(57) ABSTRACT

A prefilled syringe provides two mechanisms by which the viscosity difference between the drug-filled syringe and the placebo-filled syringe can be masked, then maintain the blindness throughout the complete clinical trials. The first mechanism is to have different thread sizes that can generate different friction forces to offset the injection force difference due to the viscosity difference between the drug and the placebo. The second mechanism is to have a plunger rod with two components, which can be assembled at different positions and generate different friction force to offset the injection force between the drug and the placebo.

### FIELD OF THE INVENTION

This invention relates to a method and system for the plunger rod in a prefilled syringe. Particularly, this invention relates to the different plunger rods that can be used with the same prefilled syringe to generate different insertion forces.

### BACKGROUND

Prefilled syringes have been widely used as a drug delivery device. Pharmaceutical companies also use prefilled syringes as the drug delivery device in their clinical studies to evaluate the safety and efficacy of drugs before commercialization. In a typical clinical study, both the drug and the placebo are filled into the same prefilled syringes and the users press either the drug or the placebo out of prefilled syringes. In order to maintain the blindness throughout the complete clinical trials, the users should not be able to tell the difference between the drug injection and the placebo injection.

Typically, there will be a viscosity difference between the drug and the placebo; the injection forces will be different between the drug and the placebo. If a user uses both the prefilled syringe with drug and the prefilled syringe with placebo, he/she will feel different pressures due to different viscosities and the blinding requirement in the clinical study will not be met.

One approach of masking the viscosity difference between the drug and the placebo is to modify the viscosity of the placebo to be equivalent to that of the drug. Beside the amount of investment and the long timeline needed to develop a viscosity-matching placebo, there are safety or toxicity concerns of new ingredients added into the placebo to match its viscosity to that of the drug. Even though the safety concern of new ingredients into viscosity-matching placebo is solved, there are other risks needed to be addressed. For example, sucrose is a common ingredient to modify the placebo's viscosity, however its potential impact to diabetics such as hyperglycemia should be considered.

The other approach to blind the viscosity difference between the drug and the placebo is to design clinical trials so that the users will only be able to use either drug-filled syringes or placebo-filled syringes. As a result, the same users will not be able to feel the pressure difference between the drug and the placebo. However, this modification of clinical trials will double the number of clinical trial users and increase the overall cost and timeline of clinical trials, which can be huge. There is still the possibility that the blindness will fail if the participants of clinical trials communicate among themselves.

Therefore, there is a need for a simple and cost-effective method to completely mask the viscosity difference between the drug and the placebo.

### BRIEF SUMMARY OF THE INVENTION

The present invention comprises a few novel plunger rod designs. One plunger rod design generally consists of at least two components. These two components can be assembled at different positions and generate different frictions between the plunger and the inside surface of the glass syringe. The injection force difference originally due to the viscosity difference between the drug and the placebo can be offset by the different frictions at different positions.

The other plunge rod design generally has different thread sizes. Placebo-filled syringes can be assembled with the plunger rod with large thread sizes; drug-filled syringes can be assembled with the plunger rod with small thread sizes. The injection force difference between the drug and the placebo can be offset by the different frictions from different thread sizes.

### BRIEF DESCRIPTION OF THE DRAWINGS

Some embodiments of the present invention are illustrated as an example and are not limited by the figures of the accompanying drawings, in which like reference may indicate similar elements and in which:

FIG. 1 depicts an exploded perspective view of elements that may comprise a prefilled syringe.

FIG. 2 shows an assembled view of a prefilled syringe.

FIG. 3 shows an assembled half-section view of a prefilled syringe with one first plunger rod embodiment.

FIG. 4 illustrates the first embodiment of plunger rod.

FIG. 5 shows a half-section view of plunger rod for the first embodiment.

FIG. 6 illustrates an exploded perspective view of the second embodiment of plunger rod.

FIG. 7 shows an assembled half-section view of the second embodiment. This plunger rod design has two components that are pre-assembled and twisted onto the plunger, which is assembled into the syringe.

FIG. 8 illustrates the plunger rod of the second embodiment.

FIG. 9 shows a half-section view of the plunger rod of the second embodiment.

FIG. 10 illustrates the plunger rod pin of the second embodiment.

FIG. 11 shows different pre-assembled positions between the plunger rod and the plunger rod pin for the second embodiment, which will lead different friction forces between the plunger and the syringe barrel.

### DETAILED DESCRIPTION OF THE INVENTION

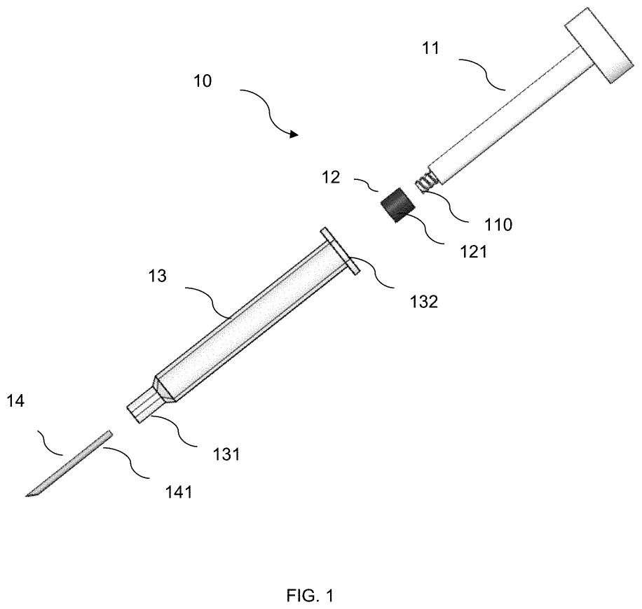

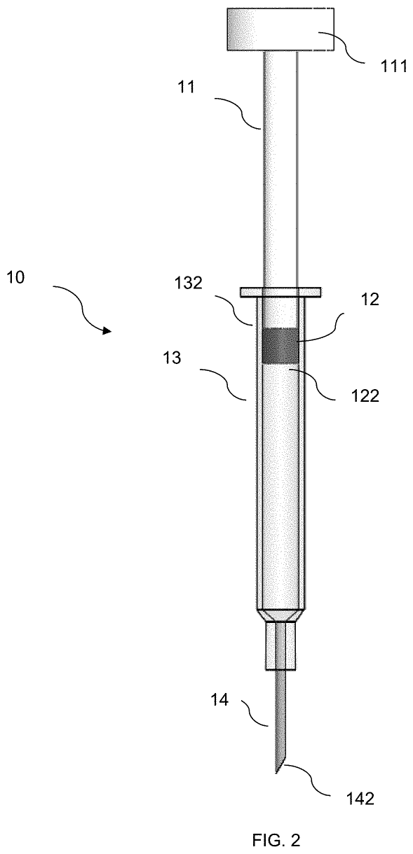

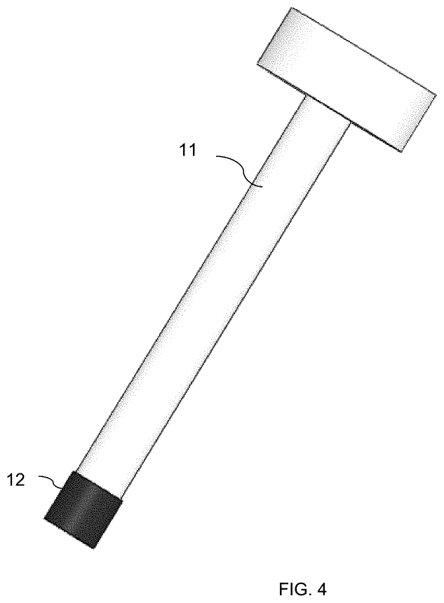

FIG. 1 shows an exploded view of a prefilled syringe assembly 10 according to the present invention which includes a syringe barrel 13, a steel needle 14, a plunger 12 and a threaded plunger rod 11. The plunger rod 11 includes a distal male threaded end 110 to facilitate connection to the proximal female threaded end 121 of plunger 12. An assembled plunger rod 11 onto the rubber plunger 12 is shown more clearly in FIG. 4. The steel needle 14 is hollow inside and its proximal end 141 is glued into the distal end 131 of syringe barrel 13. After the drug is filled from the proximal end 132 of syringe barrel 13, the distal end 122 of plunger 12 and the plunger rod 11 will be inserted from the proximal end 132 of syringe barrel 13, which is more clearly illustrated in FIG. 2. Due to the flexibility of plunger 12 materials for example rubber, it will seal the drug inside the syringe barrel and keep its sterility.

If a user starts to push the proximal end 111 of plunger rod 11, the distal end 122 of plunger 12 will start to push the drug stored in the syringe barrel 13 through the distal end 142 of needle 14. The force required to deliver the drug will depend on the factors such as the viscosity of the drug, the friction force between the outer surface of plunger 12 and the inside surface of syringe barrel 13, and the geometry of needle 14 such as the inner diameter and the length.

In many drug clinical studies, both drugs and placebos filled into prefilled syringes should be blinded so that the users will not be able to distinguish them to minimize bias and maximize the validity of the results. However, it's very common that there are significant differences between the viscosities between drugs and placebos, which leads to different delivery forces. As a result, the blinding requirement of drug clinical studies will not meet.

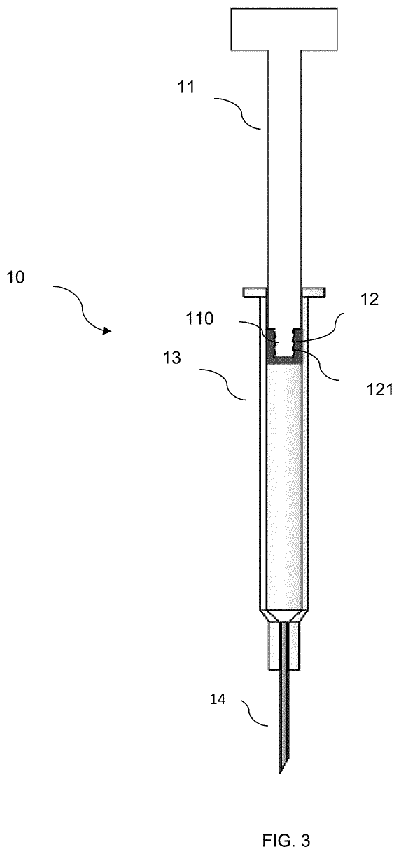

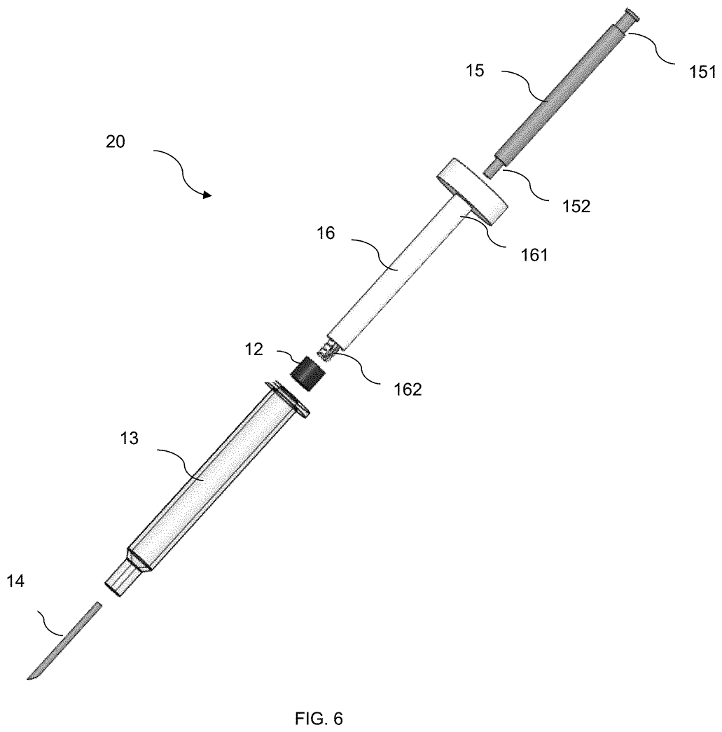

In order to address the delivery force difference of prefilled syringe between drugs and placebos, two different plunger rod designs were explained in this patent application. FIG. 3-5 explains the first plunger rod designs and FIG. 6-11 explains the second plunger rod designs.

FIG. 3 shows the half-section view of one assembled prefilled syringe embodiment with the first plunger rod design 11. The plunger rod 11 has a distal male threaded end 110 that is screwed into the female thread end 121 of plunger 12. The outer diameter of plunger 12 is slightly larger than the inner diameter of syringe barrel 13. The plunger 12 is made of rubber and flexible. It will be compressed when the subassembly of plunger rod 11 and plunger 12 is inserted into the syringe barrel 13.

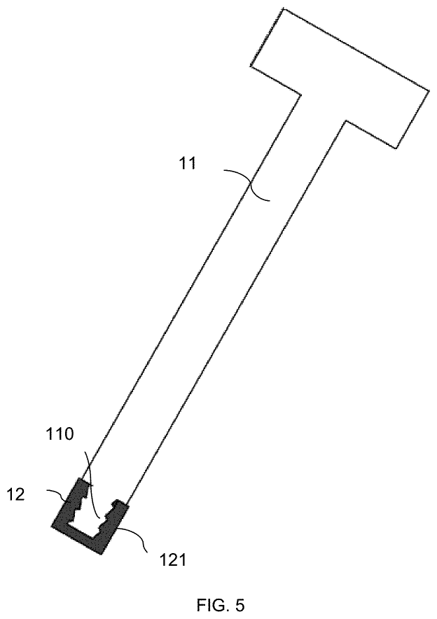

FIG. 4 shows the subassembly of plunger 12 and plunger rod 11 and FIG. 5 shows its half-section view. The plunger rod 11 will have different outer diameters of its distal male threaded end 110. The larger outer diameter of thread end 110 will compress the plunger 12 more when it is inserted into the syringe barrel 13. More compression on the plunger 12 will generate higher friction force when the plunger 12 and the plunger rod 11 slide inside of syringe barrel 13. On the other hand, the smaller outer diameters of thread end 110 will generate lower friction forces.

Even though there is the viscosity difference between the drugs and the placebos in the drug clinical studies, the different outer diameters of plunger rod 11 will be able to offset this viscosity difference. As a result, the user will have the similar injection forces between the drugs and the placebos for the blinding requirements of clinical studies.

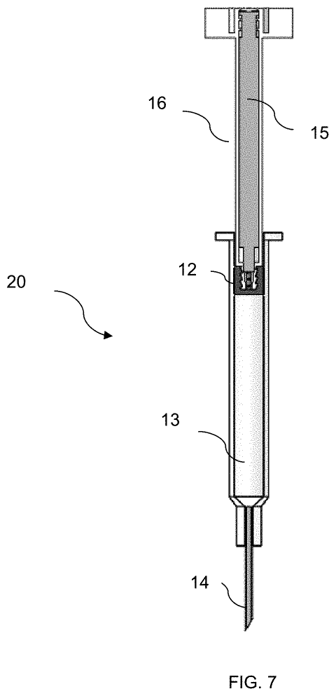

Unlike the first embodiment in which different plunger rods will be required for both drugs and placebos. FIG. 6-11 illustrates the second embodiment of plunger rod which uses the same plunger rod for both drugs and placebo. FIG. 6 shows the exploded view of prefilled syringe 20 with the second embodiment of plunger rod. The plunger rod embodiment has two components—plunger rod 16 and plunger rod pin 15. The plunger rod pin 15 is fully inserted inside of plunger rod 16 in the final assembled position, which is more clearly shown in the half-section view of assembled prefilled syringe in FIG. 7.

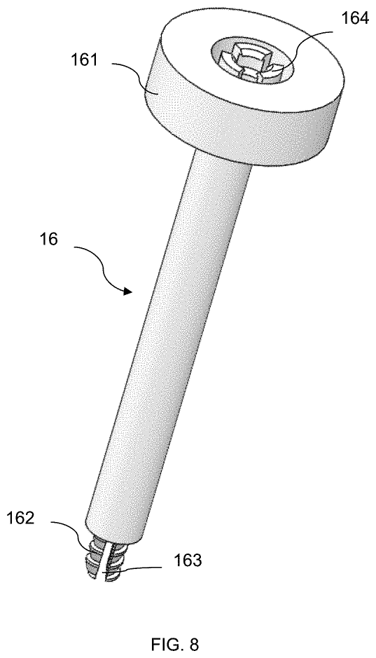

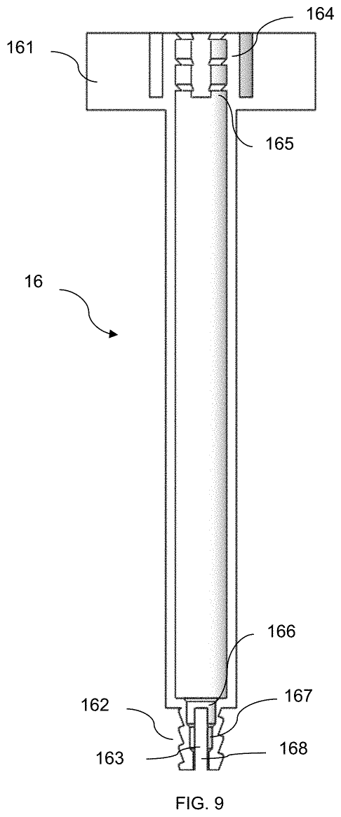

FIG. 8 shows an isometric view of plunger rod 16. It has three flexible retention fingers 164 in its proximal end 161. It also has one or multiple cuts 163 in its distal male threaded end 162 so that the thread can be slightly flexed out. These design features are more clearly shown in FIG. 9. The retention fingers 164 also has multiple inward protrusions 165. The distal end 162 has a few of hollow cavitation. FIG. 9 shows three cavitation for this embodiment. Cavitation 166 is the largest, cavitation 167 is the middle and the cavitation 168 is the smallest.

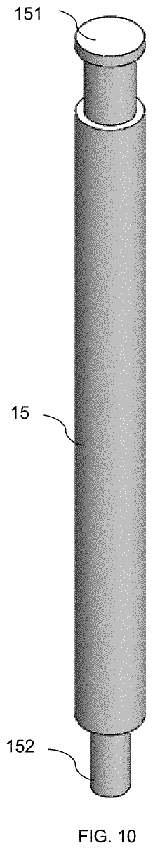

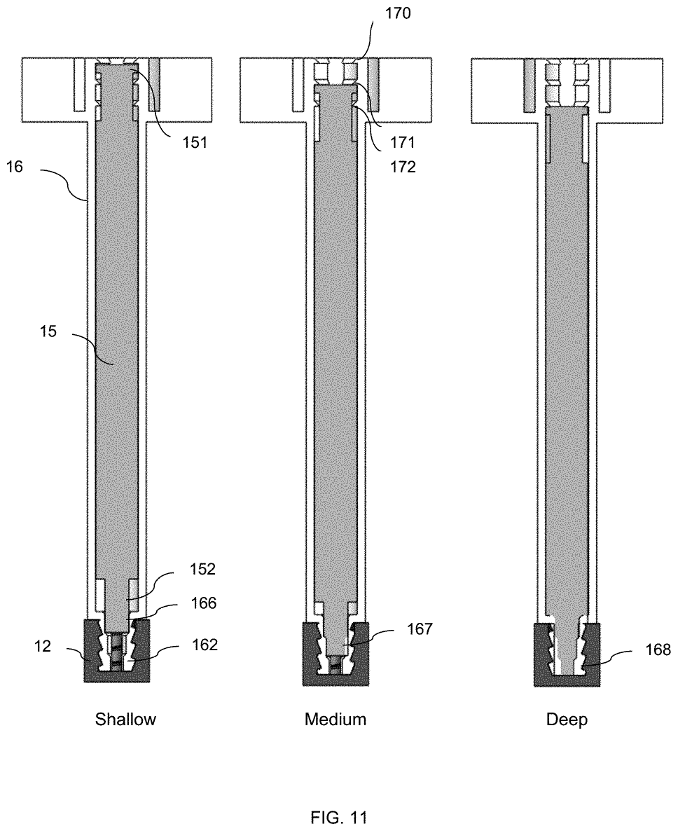

FIG. 10 shows an isometric view of plunger rod pin 15, which has a proximal end 151 and a distal end 152. FIG. 11 shows three different insertion depths (shallow, medium and deep) of plunger rod pin 15 into the plunger rod 16. The proximal end 151 of plunger rod pin 15 sit between different retention fingers of plunger rod 16 depending on the insertion depth, which also determine the location of distal end 152 of plunger rod pin 15. For the scenario of “shallow” insertion, the distal end 152 sits loosely in the largest cavitation 166 of plunger 16 so that the male threaded end 162 has not been pushed out.

For the “medium” insertion, the proximal end 151 of plunger rod pin 15 is locked between the middle retention finger 171 and the bottom finger 172. The distal end 152 of plunger rod pin is moved deeper into the smaller cavitation 167. The outer diameter of distal end 152 is larger than the inner diameter of cavitation 167 so that the male thread end 162 is pushed out and pressed onto the plunger 12. As a result, the friction between the outer surface of plunger 12 and the inner surface of syringe barrel 13 is increased compared to the “shallow” insertion. The friction force of the “deep” insertion will be the largest.

This embodiment only includes 3 different insertion depths. However, the number of insertion depths can be increased if needed. The friction force difference from different insertion depths can be used to offset the viscosity difference between the drugs and the placebos. The difference between the drugs and the placebos can be successfully blinded for the clinical studies.

This invention claimed is:

1. Pre-filled syringes with different injection forces, characterized in that they include a syringe barrel, a plunger and a plunger rod assembly;
The plunger may be connected to the plunger rod assembly, and the connection between the plunger and the plunger rod assembly may be arranged in the syringe barrel;
The plunger rod assembly is used for adjusting the radial pressure of the plunger acting on the syringe barrel to one of the pressure thresholds.

2. The prefilled syringe for generating different injection forces according to claim 1, wherein the plunger rod assembly comprises a first plunger rod and a second plunger rod;
One end of the first plunger rod is a first mounting end, one end of the second plunger rod is a second mounting end, and the diameter of the first mounting end is different from the diameter of the second mounting end;
The alternative of the first mounting end and the second mounting end is used to adjust the radial pressure of the plunger acting on the syringe barrel to one of the pressure thresholds.

3. The prefilled syringe that generates different injection forces according to claim 2, wherein the plunger is provided with a blind hole, and the direction of the blind hole is the axial direction of the plunger;
The first mounting end and the second mounting end are alternatively arranged in the blind hole.

4. The prefilled syringe for generating different injection forces according to claim 3, wherein the inner wall of the blind hole is provided with an internal thread;
The first mounting end and the second mounting end are respectively provided with external threads, and the external thread of the first mounting end and the external thread of the second mounting end are alternatively connected with the internal thread of the blind hole.

5. The prefilled syringe that generates different injection forces according to claim 1, wherein the plunger rod assembly includes a plunger rod sleeve and a plunger rod pin;
One end of the plunger rod sleeve is a deformed end, the plunger rod pin can be arranged in the plunger rod sleeve, and the plunger rod pin can be inserted into the deformed end, and the plunger rod pin can be inserted into the deformed end. The deformed end into which the pin is inserted is used to adjust the pressure of the plunger acting on the syringe barrel in the radial direction to one of the pressure thresholds.

6. The prefilled syringe that generates different injection forces according to claim 5, wherein the deformed end is provided with at least two deformed places, and a gap is formed between two adjacent deformed places;
When the plunger rod pin is inserted into the deformed end, any one of the deformed places has a tendency to expand along the radial direction of the deformed end. When the plunger rod pin moves out of the deformed end, any one of the The deformed part has a shrinking trend along the radial direction of the deformed end.

7. The prefilled syringe for generating different injection forces according to claim 6, wherein the other end of the plunger rod sleeve is a retracted end;
The backstop end has a deformed state and a non-deformed state, and the plunger rod pin is used to force the backstop end to transition between the deformed state and the non-deformed state;
When the plunger rod pin penetrates the stop end and the plunger rod pin is in a non-deformed state, the stop end is used to hinder the plunger rod pin relative to the plunger rod sleeve activity.

8. The prefilled syringe for generating different injection forces according to claim 7, wherein the inner contour of the deformed end is set as a plurality of steps, and all the steps are set to communicate coaxially, Any one of the stepped portions may respectively form an inner end surface profile, and the axial inner end surface distances of two adjacent inner end surface profiles are the same;
The stop end is configured as a plurality of deformable cantilever hooks, any one of the cantilever hooks is respectively provided with a plurality of protrusions inward along the radial direction, wherein two adjacent protrusions of any one of the cantilever hooks The distance between the axial protrusions is the same;
The axial protrusion spacing between two adjacent protrusions is the same as the axial inner end face spacing of the two adjacent inner end face profiles.

9. The prefilled syringe for generating different injection forces according to any one of claims 1 to 8, characterized in that it further comprises a hollow injection needle;
The injection needle penetrates and is fixed to the syringe barrel, wherein the two ends of the injection needle are respectively a liquid inlet end and a liquid discharge end, the liquid discharge end is located outside the syringe barrel, and the liquid inlet end Located inside the syringe barrel.

10. The liquid storage device, characterized by comprising the prefilled syringes of any one of claims 1 to 9 that produce different injection forces.
